(19) United States
(12) Patent Application Publication
(10) Pub. No.: US 2010/0331822 A1
(43) Pub. Date: Dec. 30, 2010

(54) Multi-Component Sterile Connector Assembly and Anti-Slip Cover

(76) Inventor: Benjamin Willemstyn, Little Silver, NJ (US)

Correspondence Address: GIBBONS P.C., ONE GATEWAY CENTER, NEWARK, NJ, 07102, US

(21) Appl. No.: 12/772,892

(22) Filed: May 3, 2010

Related U.S. Application Data
(60) Provisional application No. 61/175,155, filed on May 4, 2009.

(51) Int. Cl. A61M 39/10

(52) U.S. Cl. 604/533

(57) ABSTRACT

A multi-component connector apparatus having a base component, an intermediate component and an end member are assembled end to end by rotational engagement. An elastic sheath extends over and covers portions of the base member, the intermediate member and the end member in a manner that precludes inadvertent decoupling of these assembled components. The sheath also prevents contamination of the assembled components after sterilization.

### CROSS-REFERENCE TO RELATED APPLICATION

This application claims the priority of Provisional Application No. 61/175,155 filed on May 4, 2009, which is hereby incorporated by reference in its entirety.

### FIELD OF THE INVENTION

The instant invention relates to a multi-component connector assembly intended for sterile applications having a cover that protects the assembly from contamination and inadvertent disassembly.

### BACKGROUND OF THE INVENTION

Multi-component connectors designed for use in medical or pharmaceutical manufacturing environments are well known. The advantage of a multi-component connector is to provide the user of the device with several options for making a connection depending on the apparatus they are using (e.g. needle, male or female Luer syringe). The multi-component connector itself is generally made from several Luer lock type connectors which are screwed together. The multi-component connector assembly is typically attached to a larger device (e.g. single-use bioreactor, sample collection bag, etc.) via a conduit of flexible tubing. The connector is intended to retrieve a fluid sample from the larger device, or alternatively, inject material into the larger device. Multi-component connectors suffer from two primary problems when used in medical or pharmaceutical manufacturing environments. First, it is critical that the sterility of the entire multi-component connector assembly is maintained until the time it is needed. Otherwise the material being removed (or added) to the system through the multi-component connector could become contaminated. Currently, most users of these systems cover them with plastic bag material, loose fitting caps, or leave them entirely exposed to ambient conditions. The second problem with multi-component connectors is that the Luer fittings of the assembly can inadvertently disengage due to shipping and incidental handling issues. This will be catastrophic to the pharmaceutical process in that the sterility of the larger device is now fully compromised and might even leak out of the larger device completely.

Therefore, a need exists for a multi-component connector apparatus that keeps the connectors from unscrewing during shipping and handling and also maintains the sterility of the entire multi-component system.

### SUMMARY OF THE INVENTION

A multi-component connector apparatus is described. The apparatus includes a base component, an intermediate component and an end component that preferably includes an end cap. The components are assembled to one another by rotation. A sheath extends over and covers a substantial portion of the base member, the intermediate member, and the end member. The sheath precludes inadvertent decoupling of these assembled components and in applications requiring sterilized components, the sheath also prevents contamination of the assembled components after sterilization.

### BRIEF DESCRIPTION OF THE FIGURES

Understanding of the present invention will be facilitated by consideration of the following detailed description of the embodiments of the present invention taken in conjunction with the accompanying drawings, in which like numerals refer to like parts and in which:

FIG. 1 is a perspective exploded view of an exemplary connector assembly, according to the present invention;

FIG. 2 is a perspective partially exploded view of the exemplary connector assembly of FIG. 1 with components 101-104 assembled to one another and sheath 105 removed;

FIG. 3 is a perspective view of the connector assembly of FIG. 1 with an exemplary sheath 105 covering the assembly of components 101-104;

FIG. 4 is a front view of the cross-sectional view of the base unit 101 shown in FIGS. 1-3;

FIG. 5 is a cross sectional view of base unit 101 taken along section A-A of FIG. 4;

FIG. 6 is a front view of sheath 105 shown in FIG. 1;

FIG. 7 is a cross-sectional view of the sheath taken along the section A-A- of FIG. 6; and

FIG. 8 is a cross-sectional view of the sheath taken along section B-B of FIG. 6.

FIG. 9 is a perspective view of an exemplary anti-slip sheath, according to an aspect of the present invention.

### DETAILED DESCRIPTION OF THE PREFERRED EMBODIMENTS

It is to be understood that the figures and descriptions of the present invention have been simplified to illustrate elements that are relevant for a clear understanding of the present invention, while eliminating, for the purpose of clarity, many other elements found in typical multi-component mechanical connection systems. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

The present invention may generally be described as including a multi-component connector apparatus and mechanism suitable for use in a sterilized environment. The present invention further provides a sheath that prevents inadvertent contamination and disassembly of the various portions of the sterilized multi-component assembly. In an aspect of the present invention, the sheath may be open on both ends.

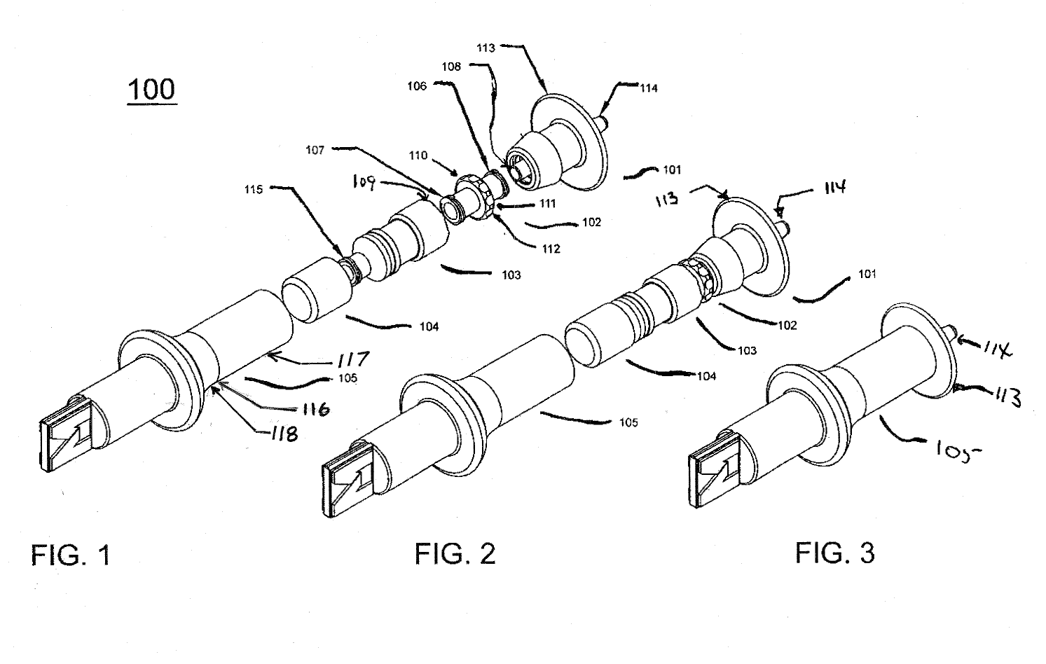

According to an aspect of the present invention and as illustrated in FIG. 1, an assembled multi-component connector assembly 100 is shown. This assembly includes a base component 101, an intermediate component 102, an end component 103, an end cap 104 and a sheath 105. Sheath 105 as shown has two portions 117 and 118 meeting at 116. Portion 118, closed on one end, facilitates handling ease of placing the sheath over the multi-component connector assembly 100 and forming a sterile covering over component 104 of multi-component connector assembly 100. Sheath portion 117 will slide over and securely hold some or all of the components of assembled multi-component connector assembly 100 in place. Sheath portion 117 may be used as a sheath without portion 118. When used alone, sheath portion 117 forms a sheath open at both ends preventing disassembly of the various portions of the sterilized multi-component assembly.

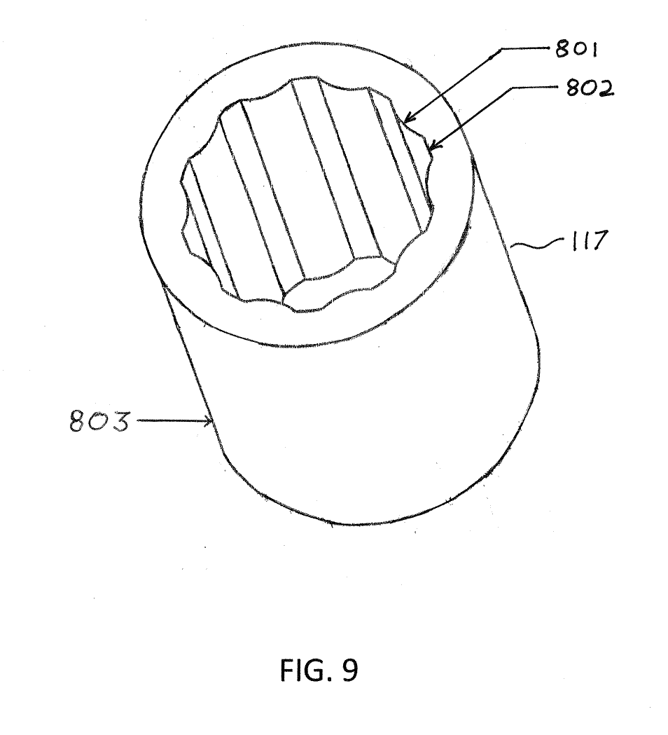

FIG. 9 is a perspective view of an exemplary anti-slip sheath portion 117, according to an aspect of the present invention. The outer surface 803 of boot 117 may be smooth, or may include a texture not shown. If present, the texture of surface 803 may be of any type or combination of texture features, such as any number or combination of knobs, ribs and/or grooves extending from all or any portion of the top surface 803 of sheath portion 117. The internal surface of sheath portion 117 may also be smooth, or may be of any type or combination of texture features, such as any number or combination of knobs, ribs and/or grooves, undulating surface extending from all or any portion of internal surface of sheath portion 117. If the internal surface is textured, the texture profile may preferentially match, or may be similar to, the texture surface profile plurality of ridges 111 of flange 110. By matching or having similar surface profiles, sheath portion 117 may provide better gripping, and effectively lock into the flange portion that boot 117 has been fitted over. Furthermore, the internal diameter of sheath portion 117 may be equal to or less the outer diameter any flange or barrel portions of the connectors. For example, the internal diameter of sheath portion 117 may be equal to or between approximately 0% and 40% less than the outer diameter of the respective connector it is fitted over. In one exemplary embodiment, the internal diameter of sheath portion 117 may be 20% less than the outer diameter of the respective connector it is fitted over. This may provide additional compression via hoop stress generated when the sheath portion is slipped or fitted over the connector components.

Sheath portion 117 may be composed of a silicon or other flexible material such that sheath portion 117 may be placed over the flange and barrel portions. Of the connector components as described herein. All or any portion of multi-component connector assembly 100 and sheath portion 117 may be sterilized.

The opposite ends of intermediate component 102 are formed with the female portions 106 and 107 of a “twist-lock” connector. For purposes of this application, the term “twist-lock connector” means a connector having mating portions that are coupled to one another after initial engagement by a twisting motion in one direction and, further, wherein such engaged components may be disengaged from one another by a twisting motion in another direction. A number of twist lock connectors are known in the art and the present invention may be used with any of these connectors. In the preferred embodiment, the twist-lock connector used is the well-known Luer connector.

As shown in FIG. 1, female portion 106 of a Luer connector in intermediate component 102 is received by a mating male portion 108 of a Luer connector disposed in base component 101. Similarly, female portion 107 of a Luer connector in intermediate component 102 is received by a mating male portion 109 of a Luer connector disposed in end component 103. Portions 106 and 108 engage with one another by first inserting portion 106 into portion 108 and then twisting intermediate component 102 in a first direction with respect to base component 101. Mating connector portions 106 and 108, once engaged, can be decoupled from one another by twisting intermediate portion 102 with respect to base portion 101 in a second direction that is opposite to the first direction. Similarly, male portion 109 of end component 103 engages with female portion 107 of intermediate component 102 by first inserting male portion 109 into female portion 107 and then twisting end component 103 with respect to intermediate component 102 in the first direction. As with mating portions 106 and 108, mating portions 109 and 107 can be decoupled from one another by twisting end component 103 in the second direction with respect to intermediate component 102.

In the preferred embodiment, intermediate component 102 with its female Luer connector portions is a commercially-available female Luer coupler. One such commercial Luer coupler is that marketed by Value Plastics, Inc. bearing part #FTLLC-9002.

As shown, intermediate component 102 also incorporates a circular flange 110 having a plurality of ridges 111 extending from the periphery of flange 110 wherein adjacent ridges are separated by a valley 112. The ridges advantageously provide a surface for gripping intermediate component 102 when engagement with or removable from base component 101 or end component 103 is desired. In the preferred embodiment of the present invention, end component 103 is Cardinal Health's Smartsite® Needleless Injection Site, Part #5000R that incorporates an internal septum (not shown) and has an end 115 configured as a female Luer connector that is covered by end cap 104 that in the preferred embodiment is Qosina Corporation's Part #12084.

As is also shown in FIG. 1, base component 101 has an end 114 formed as a hose barb to accept commercial tubing along with a circular flange 113.

Referring to FIG. 2, the components 101-103, the latter with end cap 104 inserted over end 115 of component 103 are shown assembled end to end. The assembled components provide an internal passageway from end 115 of end component 103 to end 114 of base component 101. The illustrated connector assembly is intended to provide fluid sampling by connecting conventional tubing to hose barb end 114. Fluid into which this tubing extends can be sampled by accessing the internal septum of end component 103.

FIG. 3 shows sheath 105 covering the assembly of components 101-104. As shown, sheath 105 extends over base component 101 to a position proximate to flange 113.

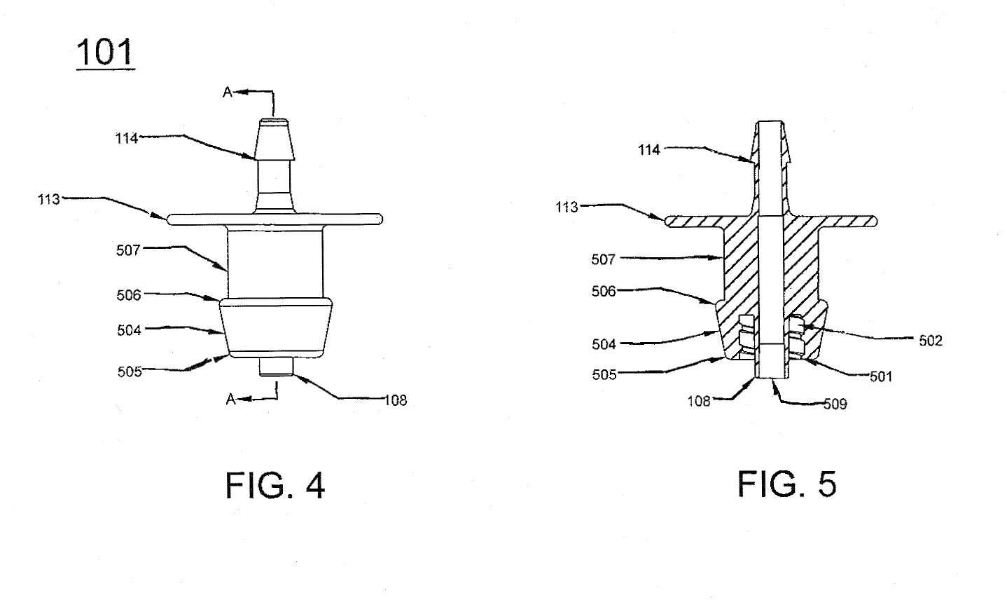

Refer now to FIGS. 4 and 5 which shows further details of base component 101. The male Luer component 108 extends from a cavity 501 having female threads 502 formed in the circumferential wall of cavity 501. The specifications of the threads are those used in a conventional Luer connector. Base component 101 also has a truncated conical portion 504 wherein the outer circumference of this conical portion increases linearly from a first diameter at end 505 to a greater second diameter at end 506. Each of ends 505 and 506 of portion 504 advantageously incorporates a radius so as to facilitate the engagement and disengagement of sheath 105 with respect to the assembled connector components 101-104. The conical shape of portion 504 serves to retain sheath 105 after it is inserted over the connector assembly as shown in FIG. 3 thereby minimizing any inadvertent contamination of a sterilized connector assembly.

Component 101 also has a barrel portion 507 extending from end 506 to circular flange 113. Flange 113 provides a surface to grip on the connector assembly when removal of sheath 105 from this assembly is desired. The diameter of barrel portion 507 is less than the diameter of conical portion 504 at end 506. A conventional hose barb end 116 extends from flange 113 in a direction away from barrel portion 507. As shown in FIG. 5, base component 101 has a passageway 509 extending completely therethrough. In the preferred embodiment, base component 101 is fabricated by molding a polycarbonate material.

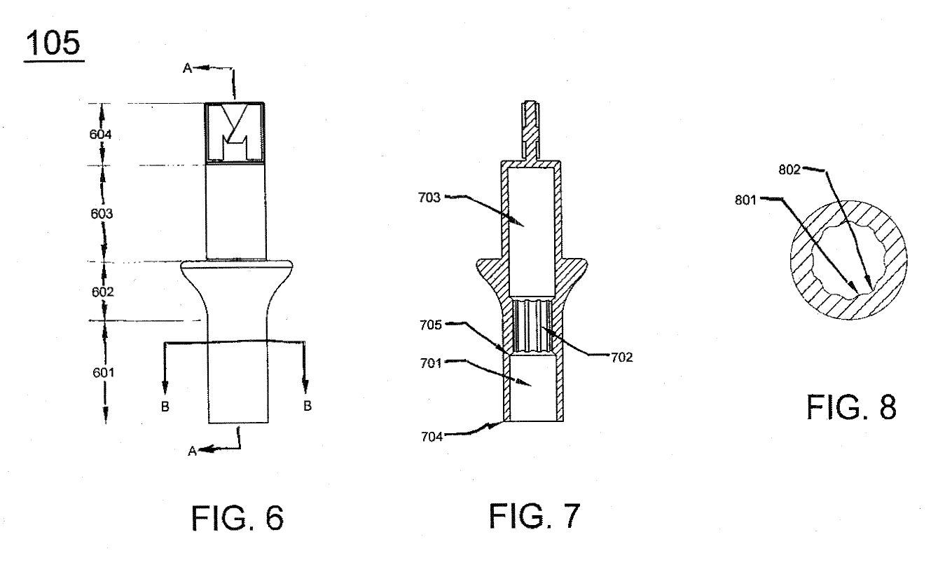

Refer now to FIG. 6. Sheath 105 includes cylindrical portion 601, expanding portion 602, first shaft portion 603 and second shaft portion 604. Portion 602 is designed for gripping of sheath 105 and second shaft portion 604 is designed placement of a logo. Internally, as shown in FIG. 7, sheath 105 includes intersecting cavities 701, 702 and 703. Cavity 701 extends from end 704 to intermediate position 705 wherein it transitions via a taper to cavity 705. Cavity 705 is formed with undulating walls that form alternating valleys and peaks that are respectively configured to engage with ridges 111 and valleys 112 of intermediate component 102 shown in FIG. 2. In this manner when sheath 105 is inserted over the assembled components 101-105, as shown in FIG. 3, the engagement of the undulating walls of cavity 702 precludes inadvertent rotation of either of mating components 101 and 102 or mating components 102 and 103. To enhance this capability, the inside diameter of cavity 702 is sized to be smaller than the outside diameter of the received intermediate component 102 and by fabricating sheath 105 from an elastic material, such as silicone rubber or Krayton elastomer. In this manner, sheath expands as necessary when inserted over components 101-105 assembled end-to-end relationship and also exerts a compressive gripping force on these covered components so as to prevent the sheath from slipping off of these assembled components. The respective engagement of ridges 111 and valleys 112 in intermediate component 102 with the valleys and peaks in undulating walls of cavity 702 in sheath 105 advantageously prevents inadvertent rotation of mating base component 101 with respect to intermediate component 102 and/or inadvertent rotation of intermediate component 102 with respect to end component 103.

Cavity 702 also transitions via a chamfer into cavity 703 that is sized to receive end component 103 and cap 104. The overall end-to-end depth of cavities 701, 702 and 703 is sized to accommodate assembled components 101,102,103 and 104 up to flange 113 of base component 101 as shown in FIG. 3. FIG. 8 shows the undulating walls of cavity 702 with its alternating peaks 801 and valleys 802 in greater detail.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

1. A sheath for a connector assembly comprising a body having serially connected first, second and third cavities extending from an end of the body, said first cavity having a first diameter, said second cavity having smooth or textured circumferential sidewalls whose maximum diameter is less than the first diameter, and said third cavity having said third diameter, said sheath being formed of an elastic material.

2. A sheath for a connector assembly of claim 1 wherein the said second cavity having undulating circumferential sidewalls.

3. A sheath for a connector assembly comprising a body having serially connected first, second and third cavities extending from an end of the body, said first cavity having a first diameter, said second cavity having smooth or textured circumferential sidewalls whose maximum diameter is less than the first diameter, and said third cavity having said third diameter, said sheath containing a connector assembly said sheath being formed of an elastic material.

4. A sheath of claim 3 wherein the second body cavity has an inner diameter less that the outer diameter of the connector assembly.

5. A sheath for a connector assembly comprising a body having a cavity having smooth or textured circumferential sidewalls said sheath being formed of an elastic material.

6. A sheath for a connector assembly comprising a body having a cavity having smooth or textured circumferential sidewalls said sheath containing a connector assembly said sheath being formed of an elastic material.

7. A sheath of claim 6 wherein the body cavity has an inner diameter less that the outer diameter of the connector assembly.
